(19) United States
(12) Patent Application Publication
(10) Pub. No.: US 2026/0108633 A1
(43) Pub. Date: Apr. 23, 2026

(54) L-ERGOTHIONEINE-GOLD NANOPARTICLES AND PREPARATION METHOD AND APPLICATION THEREOF

(71) Applicant: Guangzhou First People's Hospital, Guangzhou (CN)

(72) Inventors: Bujie DU, Guangzhou (CN); Mingze XU, Guangzhou (CN); Xingya JIANG, Guangzhou (CN)

(73) Assignee: Guangzhou First People's Hospital, Guangzhou (CN)

(21) Appl. No.: 19/212,792

(22) Filed: May 20, 2025

(30) Foreign Application Priority Data
Oct. 18, 2024 (CN) 202411455898.1

(51) Int. Cl. A61K 49/04; A61K 9/51; A61K 31/4172; A61P 13/12

(52) U.S. Cl. CPC A61K 49/04; A61K 9/5192; A61K 31/4172; A61P 13/12

(57) ABSTRACT

An application for the EGT-AuNPs in preparing contrast agents and/or antioxidants is provided, where the EGT-AuNPs include gold nanoparticles (AuNPs), the AuNPs are connected to L-ergothioneine (EGT) by gold-sulfur bonds, and the antioxidants include medicines for preventing and/or treating acute kidney injury (AKI). EGT-AuNPs with antioxidant activity are designed and synthesized through the combination of the higher imaging contrast of gold and the antioxidant natural product EGT, and they are ultra-small AuNPs, which achieve the combination of CT imaging and antioxidant effects, and are suitable for renal imaging, particularly for the early diagnosis and treatment of AKI, realizing the integration of diagnosis and treatment, circumventing the toxicity of contrast agents present in the conventional nanomaterials, lowering the serum creatinine and urea nitrogen levels, and reducing the degree of renal tubular damage.

### CROSS REFERENCE TO THE RELATED APPLICATIONS

This application is based upon and claims priority to Chinese Patent Application No. 202411455898.1, filed on Oct. 18, 2024, the entire contents of which are incorporated herein by reference.

### TECHNICAL FIELD

The present invention relates to the field of biomedical technology, particularly to L-ergothioneine-gold nanoparticles (EGT-AuNPs) and a preparation method and application thereof.

### BACKGROUND

Acute Kidney Injury (AKI) is a clinical syndrome with a rapid decline in renal function caused by a variety of etiologic factors. AKI can either occur in people without previous kidney disease or on the basis of existing chronic kidney disease. AKI seriously endangers human health, and according to the survey, about 5% of hospitalized patients may suffer from AKI, and the incidence rate of AKI in the intensive care unit (ICU) is as high as 30%. Although the nephrology community is paying more and more attention to AKI, there is no specific treatment for AKI yet, and the mortality rate is high, which makes AKI an acute and critical disease in renal diseases. Early diagnosis and treatment of AKI are extremely important to the prognosis of patients, however, the serum creatinine, which is the commonly used indicator of renal function, has a serious lag, and the creatinine level will only increase significantly when the glomerular filtration rate of the patient decreases by more than 50%, and then irreversible damage to the renal function has already happened, so it is urgently required to realize early diagnosis of AKI, and to treat timely when the renal function is less damaged, thus effectively protecting the kidney function, reducing the mortality of patients with AKI, and suppressing the transition to chronic kidney disease (CKD). During the progression of AKI, cell damage caused by intracellular oxidative stress is one of the main factors in the progression of AKI, therefore, the development of antioxidant drugs targeting reactive oxygen species (ROS) can counteract the progression of AKI and achieve effective protection of renal function.

Computed tomography (CT) technology is a widely used detection technology in clinical practice, and compared with other imaging technologies (magnetic resonance imaging (MRI), positron emission tomography (PET)), CT has low cost, rapid detection speed, and high clinical popularity; however, due to the limitation of X-ray attenuation of tissues, the imaging and diagnostic ability of CT in bones and calcification is much higher than the imaging and diagnostic ability of organs. Therefore, in order to achieve high-contrast imaging of organs, CT contrast agent for enhancement scanning has become a widely used method in clinical practice; in which, iodine contrast agent is the currently used CT contrast agent in clinical practice.

Currently, with the evolving of nanomaterials, many nanomaterials have been synthesized and applied to the diagnosis and treatment of AKI; for example, nanoprobes with fluorescent or luminescent properties have been developed through the alteration of the microenvironment of AKI, which can achieve the non-invasive detection of renal function; additionally, substances with antioxidant activity are loaded into nanomaterials, which can achieve treatment of AKI by targeting the oxidative stress of the kidneys under the condition of AKI.

Currently, the new diagnostic methods developed for the early diagnosis of AKI are mainly fluorescence imaging or urine analysis technology, but fluorescence imaging is limited by the wavelength, which is difficult to be practical application in the human body; and the urine analysis can achieve the early diagnosis of AKI, but it lacks the intuitive imaging data.

Although the use of CT iodine contrast agent can achieve enhanced imaging of kidneys, there is a risk that iodine contrast agent may cause contrast-induced renal injury due to the physicochemical properties of iodine contrast agent, the contrast-induced renal injury is a kind of drug-induced renal damage, specifically referring to acute renal failure caused by the use of contrast agent. For the elderly, diabetics, and people with pre-existing chronic kidney disease, particularly those with chronic renal insufficiency, the use of iodine contrast agent should be avoided as much as possible, therefore, it is not possible to achieve CT diagnosis of renal injury with iodine contrast agent.

### SUMMARY

An objective of the present invention is to provide L-ergothioneine-gold nanoparticles (EGT-AuNPs) with contrast agent imaging and antioxidant activity and preparation method and application thereof by overcoming the deficiencies of the existing technology.

In order to achieve the above objectives, the technical scheme adopted in the present invention is as follows:

the present invention provides an application for EGT-AuNPs in preparing contrast agents and/or antioxidants, wherein the EGT-AuNPs include gold nanoparticles (AuNPs), the AuNPs are connected to L-ergothioneine (EGT) by gold-sulfur bonds, and the antioxidants include medicines for preventing and/or treating acute kidney injury (AKI).

The EGT-AuNPs of the present invention are ultra-small gold nanoparticles with a contrast imaging effect and an antioxidant effect, and are particularly suitable for renal contrast imaging, particularly for early diagnosis and treatment of AKI, so as to achieve an integration of diagnosis and treatment.

Further, the contrast agent is a renal contrast agent.

In a specific embodiment of the present invention, the drug reduces serum creatinine and urea nitrogen levels, and reduces a degree of renal tubular damage.

Further, an average particle diameter of the EGT-AuNPs is 1.516-3.210 nm.

Further, a hydrated particle diameter of the EGT-AuNPs is 1.726-3.462 nm.

Further, the EGT-AuNPs are spherical or spherical-like nanoparticles.

Further, a preparation method for the EGT-AuNPs is as follows: reacting an EGT alkaline solution with a tetrachloroauric acid solution to obtain the EGT-AuNPs.

Further, a molar ratio of the tetrachloroauric acid to EGT is 1:(0.7-1.5), preferably 1:0.7. The ultra-small AuNPs can be synthesized when the molar ratio of tetrachloroauric acid to EGT is 1:(0.7-1.5), but the ultra-small AuNPs cannot be synthesized when the molar ratio of tetrachloroauric acid to EGT is 1:0.5 and 1:2.

Further, a concentration of the EGT is 0.2-1.0 mg/mL, preferably 0.55 mg/mL.

Further, a concentration of tetrachloroauric acid is 0.5-1.5 M, preferably 1 M.

Further, a reaction temperature is 50-100° C., preferably 70° C.

Further, a stirring speed is 200-1000 rpm, preferably 420 rpm.

Further, a reaction time is 5-20 h, preferably 15 h. When the reaction time is 15 h, a fluorescence intensity of the nanoparticles prepared by EGT-AuNPs is the highest.

Further, a pH value of the EGT alkaline solution is 8-12, preferably 12. A reagent for adjusting the pH of the EGT alkaline solution can be at least one of NaOH, KOH, aqueous ammonia, and Na2CO3, and in a specific embodiment of the present invention, the used reagent is NaOH.

Further, after the reaction, the EGT-AuNPs are purified.

In a specific embodiment of the present invention, a purification method is an ultrafiltration centrifuge tube purification.

Further, the ultrafiltration centrifuge tube with a retention of 1000-5000 daltons is used for purification, preferably 5000 daltons.

Further, a number of purification times are 1-3 times, preferably 3 times.

Compared with the existing technology, the beneficial effects of the present invention are: compared with iodine, gold has a higher atomic number and a larger relative atomic mass, so gold has a higher attenuation coefficient of X-rays under the same molar concentration; the present invention designs and synthesizes EGT-AuNPs with antioxidant activity through the combination of the higher imaging contrast of gold and the antioxidant natural product EGT, and they are ultra-small AuNPs, which achieve the combination of CT imaging and antioxidant effects, and are suitable for renal imaging, particularly for the early diagnosis and treatment of AKI, realizing the integration of diagnosis and treatment, circumventing the toxicity of contrast agents present in the conventional nanomaterials, lowering the serum creatinine and urea nitrogen levels, and reducing the degree of renal tubular damage.

As a natural product widely existed in food, EGT has a good guarantee of safety, and the ultra-small AuNPs have excellent renal targeting ability, which can increase the enrichment of the material in the kidneys and reduce the non-specific uptake of other organs, so as to increase the therapeutic efficacy and reduce the side effects at the same time.

### BRIEF DESCRIPTION OF THE DRAWINGS

FIGS. 1A-1B are synthesis principle and an appearance of EGT-AuNPs. Wherein FIG. 1A is the synthesis principle; and FIG. 1B is the appearance.

FIG. 2 is an ultraviolet-visible (UV-Vis) spectrophotometer spectra of EGT-AuNPs prepared with different ratios of tetrachloroauric acid and EGT.

FIG. 3 is a fluorescence spectra of EGT-AuNPs prepared with different reaction times.

FIG. 4 is a core diameter of EGT-AuNPs.

FIG. 5 is a hydrodynamic diameter of EGT-AuNPs.

FIG. 6 is an excitation/emission spectrum and a UV-Vis absorption spectrum of EGT-AuNPs.

FIG. 7 is an in vitro antioxidant activity of EGT-AuNPs.

FIG. 8 is an in vitro CT signal intensity of EGT-AuNPs and Iopromide.

FIGS. 9A-9B are imaging effects of EGT-AuNPs in mice in vivo (0-30 min). Wherein FIG. 9A is a bladder area; and FIG. 9B is a kidney area.

FIGS. 10A-10B are imaging effects of Iopromide and EGT-AuNPs in mice in vivo (0-10 min). Where FIG. 10A is Iopromide; and FIG. 10B is EGT-AuNPs.

FIGS. 11A-11C are CT images of the kidney in normal and AKI mice models. Wherein FIG. 11A is a blank image without injection of EGT-AuNPs; FIG. 11B is an image 10 min after injection of EGT-AuNPs; and FIG. 11C is an image after injection of EGT-AuNPs for 30 min.

FIG. 12 is a signal distribution of kidney profiles in normal and AKI mice models after injection of EGT-AuNPs for 10 min.

FIGS. 13A-13B are serum creatinine and urea nitrogen levels in the AKI mice model after treatment with EGT-AuNPs. Wherein FIG. 13A is serum creatinine; and FIG. 13B is urea nitrogen.

FIGS. 14A-14B are renal histopathological sections and tubular injury scores of AKI mice treated with EGT-AuNPs. Wherein FIG. 14A is a pathologic section; and FIG. 14B is a tubular injury score.

### DETAILED DESCRIPTION OF THE EMBODIMENTS

The following is a further description of the present invention in relation to specific embodiments in order to further illustrate the objects, technical solutions and advantages of the present invention. Unless otherwise specified, other materials, reagents and the like used in the embodiments are commercially available.

### Embodiment 1 Preparation for EGT-AuNPs with Antioxidant Activity

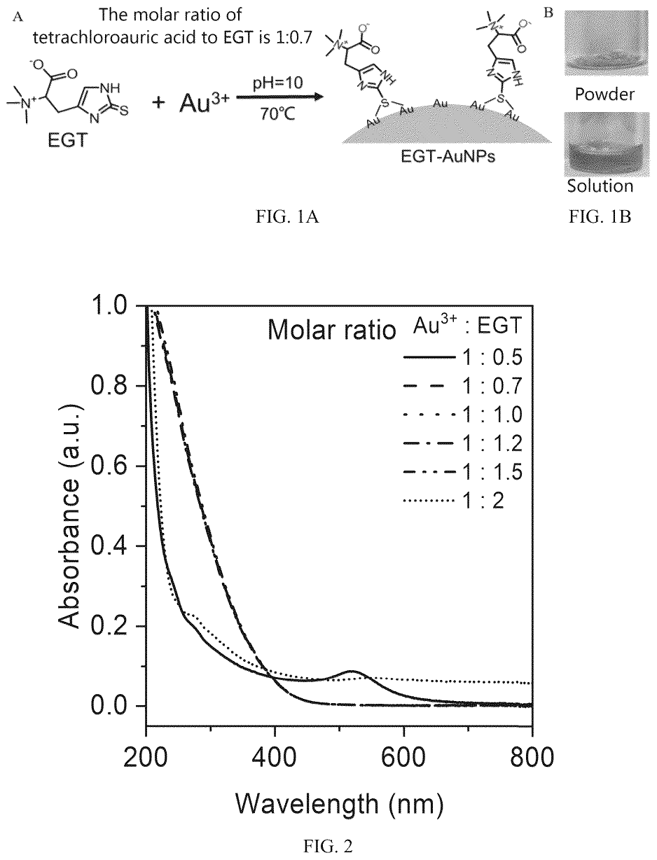

As shown in FIGS. 1A-1B, FIG. 1A is the synthesis principle of EGT-AuNPs, FIG. 1B is the appearance of EGT-AuNPs powder and solution in the form of ultra-small spherical nanoparticles.
1. 27.5 mg of EGT was dissolved in 50 mL of 0.1 mM NaOH aqueous solution, 150 μL of 1M tetrachloroauric acid aqueous solution was rapidly added at 70° C. and 420 rpm stirring speed, and the reaction was maintained at 70° C. and 420 rpm stirring speed for 15 h to obtain the reaction solution. The molar ratio of tetrachloroauric acid to EGT was 1:0.7.
2. The reaction solution of step 1 was reduced to room temperature, and purified 3 times with the ultrafiltration centrifuge tube with a retention of 5000 daltons to obtain a retentate; the retentate was freeze-dried to obtain EGT-AuNPs.

### Embodiment 2 Effect of Different Ratios of Tetrachloroauric Acid and EGT and Reaction Time on EGT-AuNPs

### I. Experimental Method

1. The molar ratios of tetrachloroauric acid and EGT in the reaction system were set as 1:0.5, 1:0.7, 1:1.0, 1:1.2, 1:1.5 and 1:2.0, respectively, the above different ratios correspond to the volume of 100 μL, 150 μL, 200 μL, 250 μL, 300 μL and 400 μL of 1 M tetrachloroauric acid solution added in turn, the other parameters and methods for preparing EGT-AuNPs were the same as those of embodiment 1, the EGT-AuNPs were prepared, and the EGT-AuNPs were characterized by UV-Vis spectrophotometer. Whether there are large-sized nanoparticles is judged by if there is an absorption peak at 500-600 nm, and large-sized nanoparticles will produce absorption at 500-600 nm. Particle size≤3 nm is ultra-small particles.
2. The reaction time was set at 5 h, 10 h, 15 h and 20 h, respectively, the other parameters and methods for preparing EGT-AuNPs were the same as those of embodiment 1, the EGT-AuNPs were prepared and the fluorescence properties of EGT-AuNPs were characterized by a fluorescence spectrometer (FL).

### II. Results of the Experiment

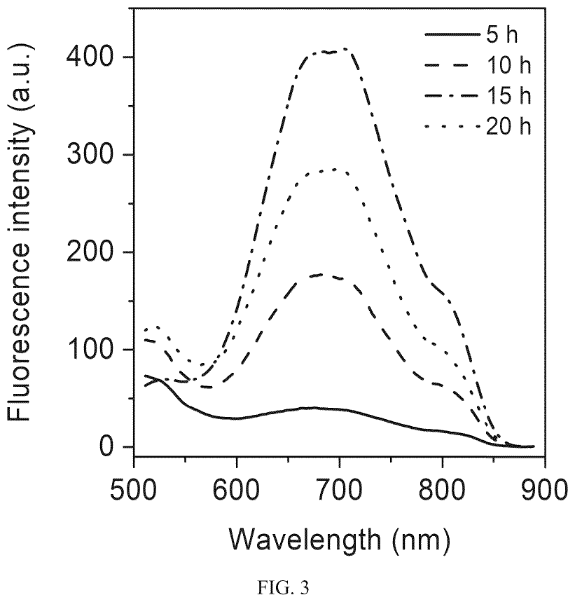

1. As shown in FIG. 2, ultra-small AuNPs can be synthesized in the range of 1:(0.7-1.5) for molar ratios of tetrachloroauric acid to EGT, but ultra-small AuNPs cannot be synthesized when the molar ratios of tetrachloroauric acid to EGT is 1:0.5 and 1:2.
2. As shown in FIG. 3, when the reaction time is 15 h, the fluorescence intensity of the nanoparticles prepared by EGT-AuNPs is the highest, so the optimal reaction time is 15 h.

### Embodiment 3 Physicochemical Properties of EGT-AuNPs

### I. Experimental Method

1. The diameter and morphology of EGT-AuNPs prepared by embodiment 1 were characterized by transmission electron microscopy (TEM), and the average diameter was calculated as the average particle size (core diameter) of EGT-AuNPs; the hydrodynamic diameter of EGT-AuNPs prepared by embodiment 1 was characterized by dynamic light scattering (DLS), and the average value was calculated as the hydration particle size of EGT-AuNPs; the characteristic absorption of EGT-AuNPs prepared by embodiment 1 was characterized by UV-Vis spectrophotometer; the fluorescence properties of EGT-AuNPs prepared by embodiment 1 were characterized by FL.
2. The antioxidant activity of EGT-AuNPs prepared by embodiment 1 was detected by 2,2′-azino-bis(3-ethyl-benzothiazole-6-sulfonic acid) diammonium salt (ABTS) free radical scavenging experiment, and the method was as follows:
(1) ABTS was dissolved in sodium acetate solution with a pH value of 4.5, the ABTS solution with a concentration of 7.4 mmol/L was prepared, and the ABTS solution was mixed with 2.6 mmol/L potassium persulfate solution at a volume ratio of 1:1, and the reaction solution was obtained by standing in the dark for 12 h at room temperature.
(2) Detection method: different concentrations (0.1 mg/mL, 0.5 mg/mL, 1 mg/mL, 2.5 mg/mL and 10 mg/mL) of EGT-AuNPs phosphate buffer solution (PBS) were mixed with the reaction solution of step (1) at a volume ratio of 1:9, after reaction for 30 min in the dark, the absorbance was detected at 734 nm, and the total antioxidant activity at different concentrations was calculated by zeroing with distilled water

### II. Results of the Experiment

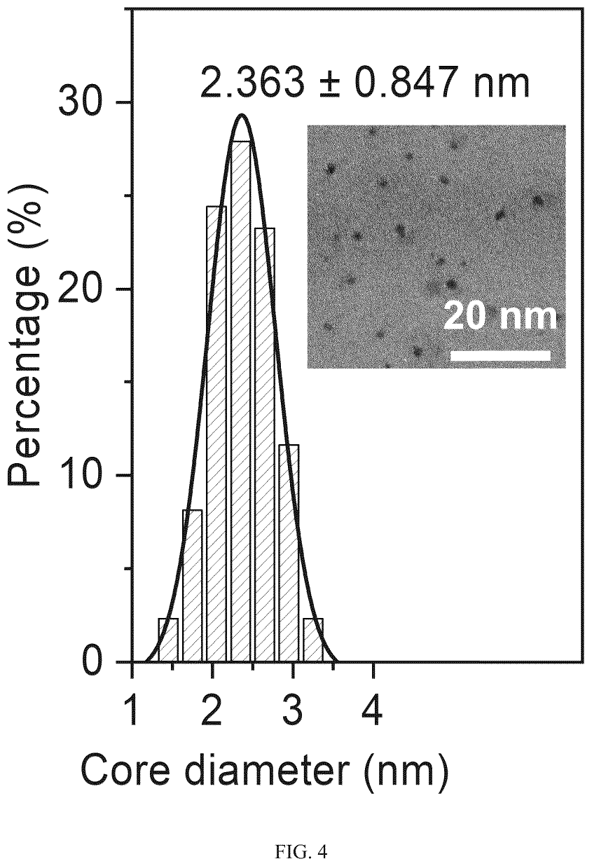

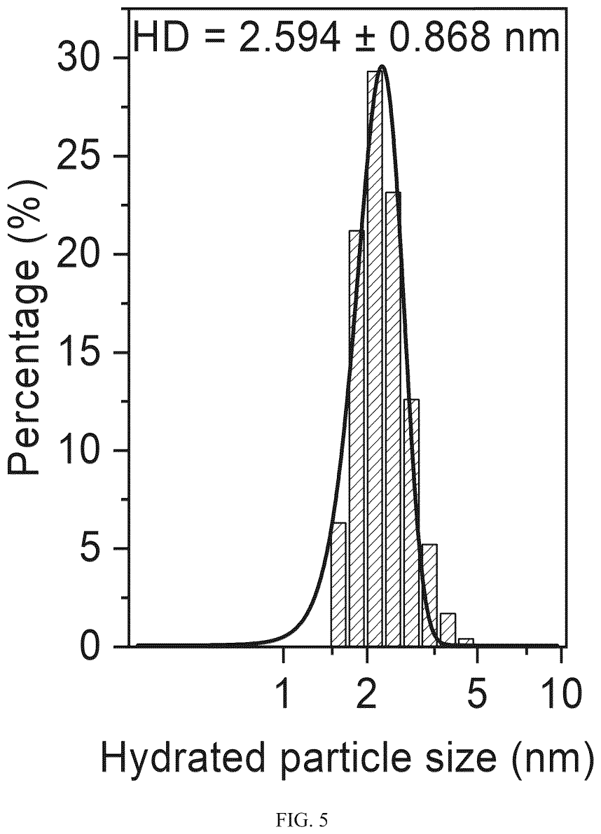

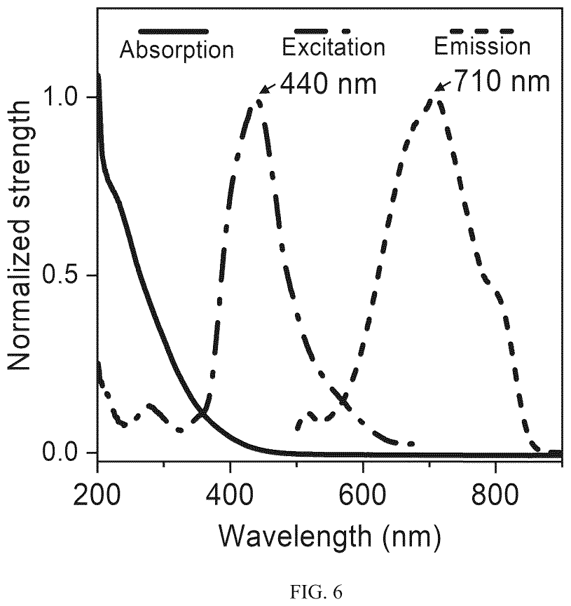

1. (1) As shown in FIG. 4, the core diameter of EGT-AuNPs prepared by embodiment 1 is 2.363±0.847 nm, which is spherical and consistent with the size range of ultra-small AuNPs.
(2) As shown in FIG. 5, the hydrated particle size of EGT-AuNPs prepared by embodiment 1 is 2.594±0.868 nm, which is consistent with the size range of ultra-small AuNPs.
(3) As shown in FIG. 6, the EGT-AuNPs prepared by embodiment 1 have fluorescence properties, the optimal excitation wavelength is 460 nm, and the maximum emission wavelength is 710 nm. Within the range of 400-500 nm, there is no obvious UV-Vis absorption in the nanoparticles, which proved that there is no surface plasmon resonance and belongs to ultra-small nanoparticles.

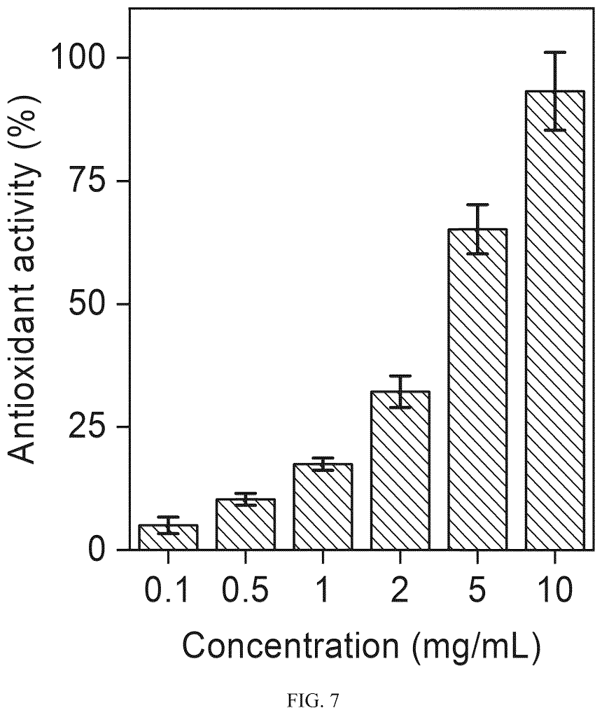

As shown in FIG. 7, the EGT-AuNPs prepared by embodiment 1 have good antioxidant activity.

### Embodiment 4 Imaging Ability of EGT-AuNPs

### I. Experimental Method

The CT imaging ability of EGT-AuNPs prepared by embodiment 1 was characterized in vitro and in vivo by using a small animal in vivo tomography imaging system (Micro-CT, Quantum GX2, manufacturer: PerkinElmer).
1. The EGT-AuNPs solution with a concentration of 100 mg/mL was prepared with PBS (prepared by embodiment 1, the concentration of gold was 20 mM). 100 μL of EGT-AuNPs solution was added to 96-well plates, and PBS-configured iodine contrast agent solution (Iopromide, the concentration of iodine was 20 mM) and PBS were used as controls, the difference in signal values under the same amount of gold and iodine was compared to clarify the CT enhanced imaging ability of EGT-AuNPs.
2. (1) 200 μL of EGT-AuNPs solution, iodine contrast agent solution and PBS were injected into the tail vein of mice.
(2) CT scan was performed with Micro-CT, the scanning parameters were 90 kV and the scanning time was 2 min, blank scan was performed before the injection of contrast agent to obtain unenhanced images of kidney and bladder regions, after injection of contrast agent, 15 consecutive scans were performed for a total of 30 min to obtain images of kidney and bladder regions.
(3) In order to compare the difference in imaging effect between the two contrast agents EGT-AuNPs and Iopromide, the signal intensity of renal pelvis and renal parenchyma was circled by CT image analysis software Analyzer, and the imaging enhancement effect of the two contrast agents in the whole renal region, renal pelvis and renal parenchyma was compared.

### II. Results of the Experiment

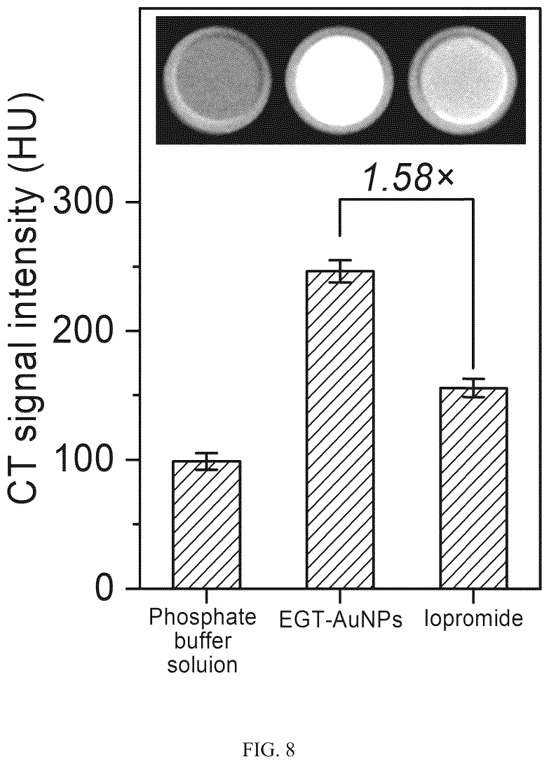

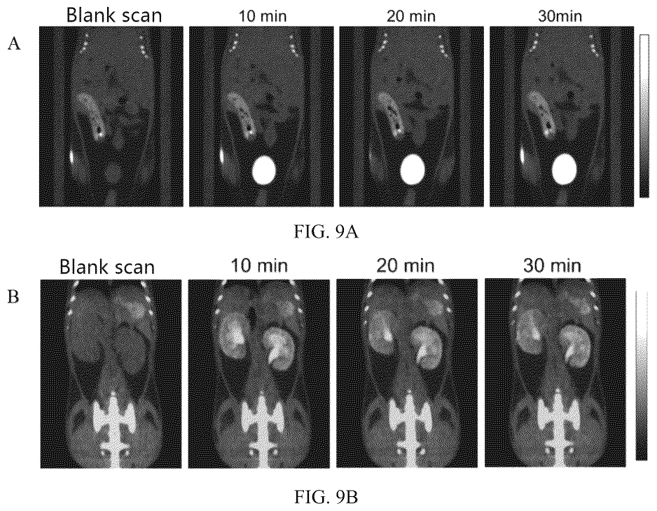

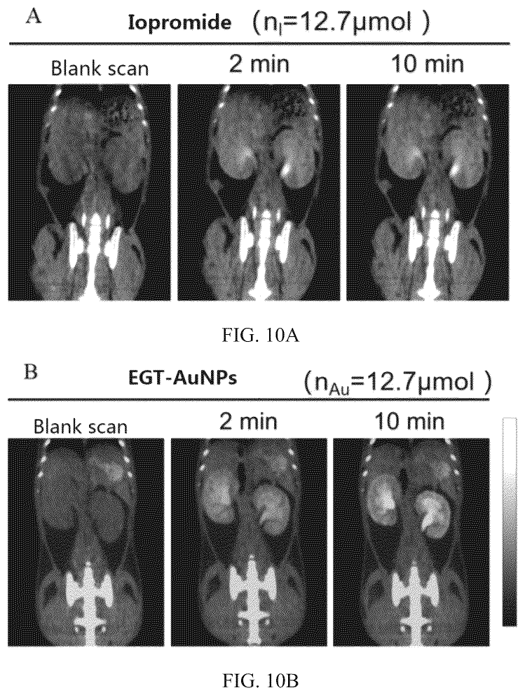

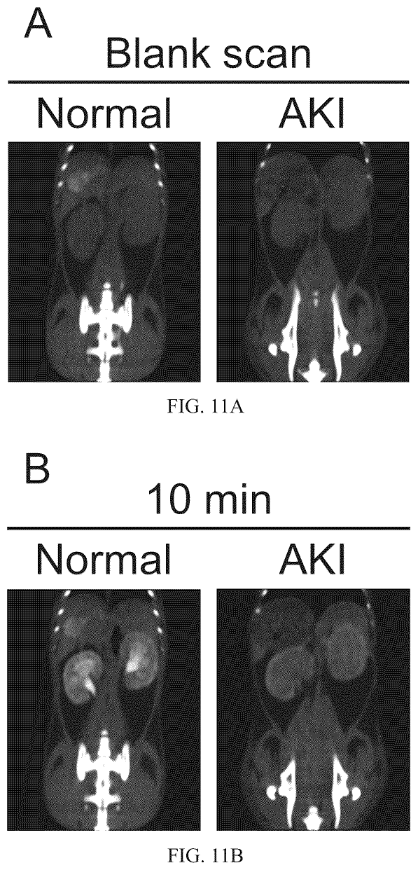

1. As shown in FIG. 8, EGT-AuNPs have a signal intensity 1.58 times higher than the signal intensity of Iopromide.
2. As shown in FIG. 9A and FIG. 9B, EGT-AuNPs effectively enhanced the signal intensity of the kidney and bladder regions and maintained the imaging contrast of the kidney and bladder within 30 min after injection.
3. As shown in FIG. 10A and FIG. 10B, compared with Iopromide, EGT-AuNPs had better renal enhanced imaging with clearer renal structure.

### Embodiment 5 Diagnostic and Therapeutic Effects of EGT-AuNPs on AKI

### I. Experimental Method

The AKI mice model of rhabdomyolysis-induced was induced and obtained by intramuscular injection of 50% glycerol at a dose of 8 mL/kg into the lower limbs of mice, the kidney CT scan was performed after 2 h of the glycerol injection to obtain blank scan images, the EGT-AuNPs prepared by embodiment 1 were injected into the mice model at a dose of 200 mg/kg through the tail vein, and Micro-CT was used for consecutive scanning at 2 min/time for a total of 15 times. The difference between renal imaging under normal and AKI conditions was compared, and the signal intensity of the kidney region was analyzed by using software to perform a non-invasive diagnosis of AKI.
2. After 24 h of EGT-AuNPs injection, the biochemical indexes of blood and kidney function (creatinine, and urea nitrogen) and renal pathological sections of the mice model were detected to determine the degree of AKI and the therapeutic effect.

After 24 h of EGT-AuNPs injection, the mice model was anesthetized with isoflurane gas, and blood was taken from the heart by puncture, the serum was separated after natural coagulation, and the levels of creatinine and urea nitrogen in serum were detected by automatic biochemical analyzer. The abdomen was opened longitudinally along the middle of the abdomen. After the kidney was taken out, the tissue was fixed with 4% paraformaldehyde, embedded in paraffin to obtain the paraffin sections of the kidney, and hematoxylin and eosin (H&E) staining was performed to analyze the degree of renal tubular injury, ten high-power fields were randomly selected for each kidney section, and scored based on to the following criteria: 1 point for the obvious expansion of renal tubules and cell flattening; 1 point for brush edge damage, 2 point for shedding; 2 point for the tube type, 1 point for the shedding and necrotic cells (not forming tube type or cell debris) in the renal tubular lumen; and the highest score of each field of vision is 4 points, and the lowest score is 0 points.

### II. Results of the Experiment

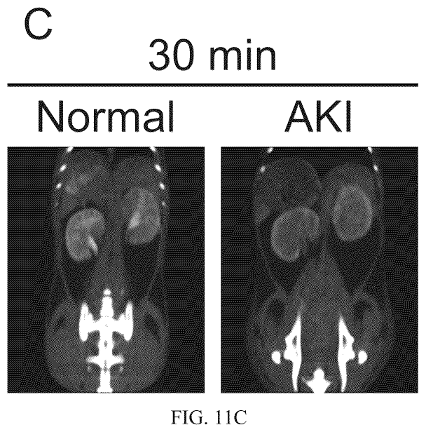

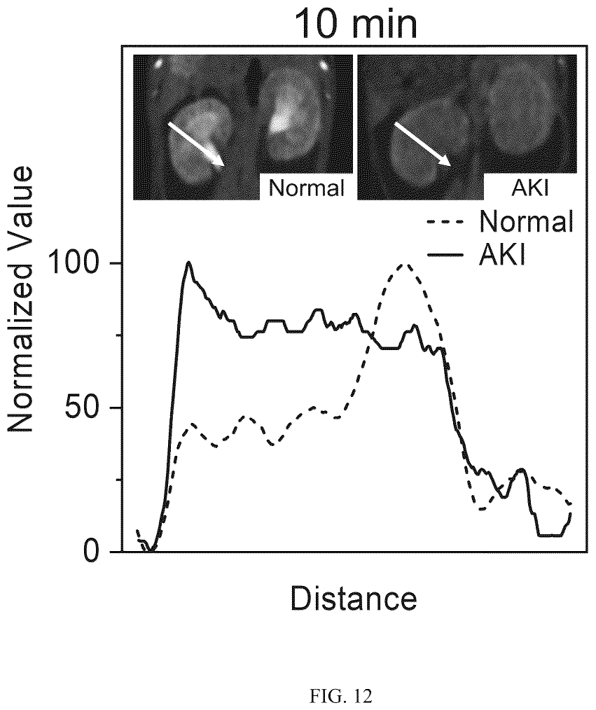

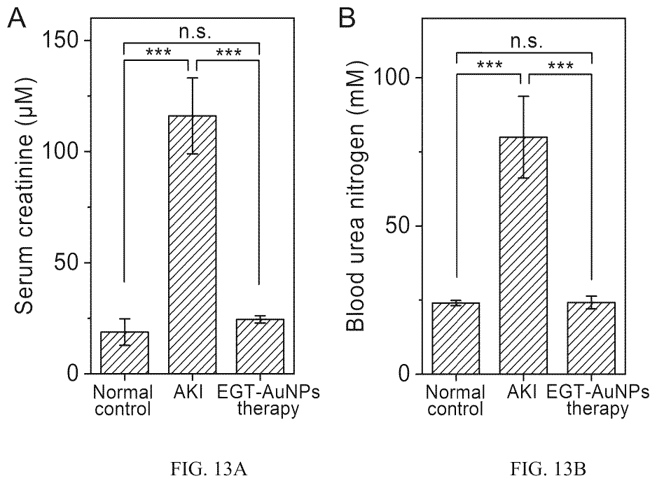

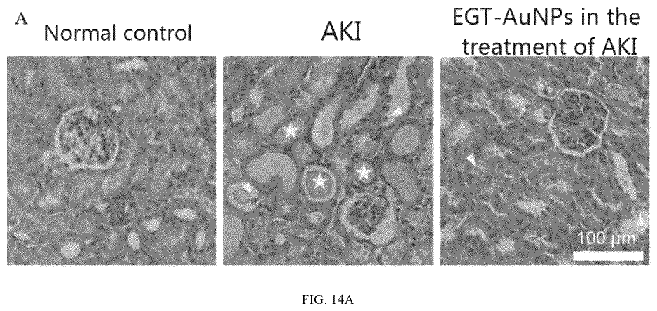

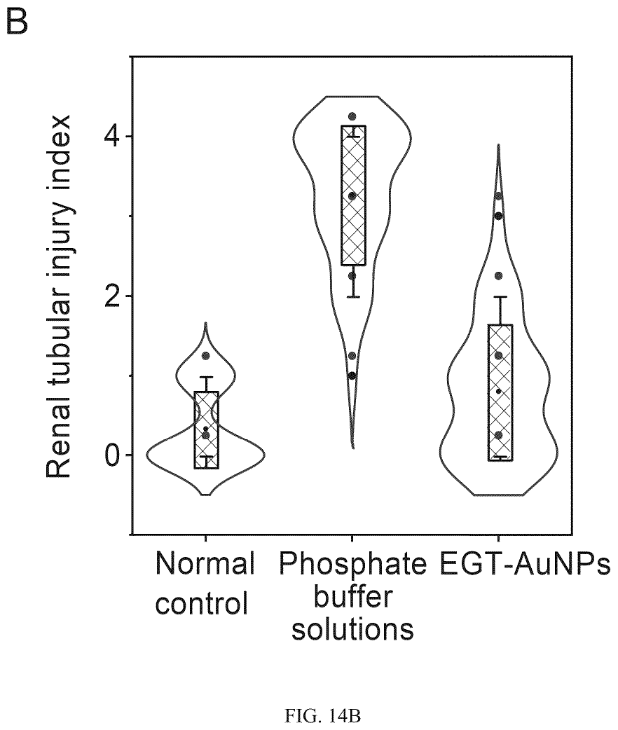

1. As shown in FIGS. 11A-11C, in the early stage of AKI (after 2 h of glycerol intramuscular injection), compared with normal mice, the renal cortex signals of AKI mice were more obvious, and the renal pelvis and medulla signals were lower. Compared with 30 min (FIG. 11C) and 10 min (FIG. 11B) after injection of EGT-AuNPs, the mice continued to maintain obvious signals in the AKI condition, and the overall signal intensity of the kidneys of normal mice decreased.
2. As shown in FIG. 12, after 10 min of EGT-AuNPs injection, there was a significant difference in the signal distribution of the kidney section (from the cortex to the renal pelvis) between normal and AKI conditions.
3. After 24 h of treatment with EGT-AuNPs, the serum creatinine (FIG. 13A) and urea nitrogen levels (FIG. 13B) of mice treated with EGT-AuNPs were significantly lower than those of untreated mice and returned to normal levels.
4. As shown in FIG. 14A and FIG. 14B, H&E staining results showed that the degree of renal tubular injury in mice treated with EGT-AuNPs was significantly reduced.

Finally, it should be noted that the above examples are merely used for describing the technical solutions of the present invention, rather than limiting the same. Although the present invention has been described in detail with reference to the preferred examples, those of ordinary skill in the art should understand that the technical solutions of the present invention may still be modified or equivalently replaced. However, these modifications or substitutions should not make the modified technical solutions deviate from the spirit and scope of the technical solutions of the present invention.

What is claimed is:

1. A method for preparing a contrast agent and/or antioxidants, comprising using L-ergothioneine-gold nanoparticles (EGT-AuNPs), wherein the EGT-AuNPs comprise gold nanoparticles (AuNPs), the AuNPs are connected to L-ergothioneine (EGT) by gold-sulfur bonds, and the antioxidants comprise medicines for preventing and/or treating an acute kidney injury (AKI);
wherein the contrast agent is a renal contrast agent;
a preparation method for the EGT-AuNPs comprises: reacting an EGT alkaline solution with a tetrachloroauric acid solution to obtain the EGT-AuNPs; and
a molar ratio of tetrachloroauric acid in the tetrachloroauric acid solution to the EGT in the EGT alkaline solution is 1:(0.7-1.5).

2. The method according to claim 1, wherein an average particle diameter of the EGT-AuNPs is 1.516-3.210 nm.

3. The method according to claim 1, wherein a hydrated particle diameter of the EGT-AuNPs is 1.726-3.462 nm.

4. The method according to claim 1, wherein the EGT-AuNPs are spherical nanoparticles or spherical-like nanoparticles.

5. The method according to claim 1, wherein a reaction time is 5-20 h.
